(19) United States
(12) Patent Application Publication
(10) Pub. No.: US 2025/0352779 A1
(43) Pub. Date: Nov. 20, 2025

(54) LUER FITTING

(71) Applicant: Johnson & Johnson Surgical Vision, Inc., Irvine, CA (US)

(72) Inventors: Chetan Nirkhe, Aliso Viejo, CA (US); Mark Ross, Costa Mesa, CA (US); Scott Strayer, Rancho Santa Margarita, CA (US); Daniel Kao, Lake Forest, CA (US)

(21) Appl. No.: 18/664,988

(22) Filed: May 15, 2024

(51) Int. Cl. A61M 39/10

(52) U.S. Cl. CPC A61M 39/10; A61M 2039/1083; A61M 2039/1088

(57) ABSTRACT

A luer fitting that included a body having a proximal region, a distal region, and a length therebetween. The body includes: a recessed region within a portion of the length of the body at the proximal region, the recessed region having a proximal end opening, an inner back wall and an inner circumferential sidewall; a barb extending proximally from the inner back wall; and a passage between a first opening at the distal region of the body to a second opening at a proximal end of the barb, the passage forming an inner surface and having a first inner diameter at or near the first opening at the distal region opening that is greater than a second inner diameter at or near the inner back wall of the recessed region, wherein the transition between the first inner diameter and the second inner diameter is curvilinear.

### FIELD OF THE DISCLOSURE

This disclosure relates generally to luer fittings, and more particularly luer fittings for phacoemulsification handpieces.

### BACKGROUND

A cataract is a clouding and hardening of the eye's natural lens. Phacoemulsification cataract surgery is a common procedure to treat a cataract. In this procedure, a clear cornea or scleral incision is made in the eye to access its internal structures, and a portion of the anterior surface of the lens capsule is removed to gain access to the cataract. The surgeon then inserts a needle of an ultrasonic handpiece into the cataract. The tip of the needle vibrates at an ultrasonic frequency to emulsify the cataract lens. Simultaneously, a pump aspirates emulsified particles and fluid from the eye through the tip of the needle, and the aspirated fluids are replaced with an irrigation of a balanced salt solution via a sleeve of the handpiece to maintain intraocular pressure in the anterior chamber of the eye.

The handpiece and tubing are typically connected via a luer to ensure a proper seal, thereby creating a flow path to aspirate the emulsified particles and fluid from the handpiece to the pump. Unfortunately, emulsified particles tend to clog within the lumen of conventional luers. To unclog the aspiration line, the luer and tubing must typically be separated from one another to clear any blockages. Because conventional luers are also difficult to grip, there is a need for an improved luer that reduces clogging and permits a more efficient engagement and disengagement process.

### BRIEF DESCRIPTION OF THE DRAWINGS

The following drawings are illustrative of particular examples of the present disclosure and therefore do not limit the scope of the present disclosure. The drawings are not to scale and are intended for use in conjunction with the explanations in the following detailed description. In the drawings, like reference numerals may represent like parts and assemblies throughout the several views.

FIG. 1 illustrates an example handpiece of a phacoemulsification system.

FIG. 2A illustrates a perspective view of an example luer.

FIG. 2B illustrates a side view of the example luer of FIG. 2A.

FIG. 2C illustrates a cross-sectional side view of the example luer taken along section A-A illustrated in FIG. 2B.

FIG. 2D illustrates a rear view of the example luer of FIG. 2A.

FIG. 2E illustrates another side view of the example luer of FIG. 2A.

FIGS. 3A and 3B illustrate perspective views of another example luer.

FIG. 3C illustrates a rear view of the example luer of FIGS. 3A and 3B.

FIG. 3D illustrates a side view of the example luer of FIGS. 3A and 3B.

FIG. 3E illustrates a cross-sectional side view of the example luer taken along section B-B illustrated in FIG. 3D.

FIG. 3F illustrates a cross-sectional side view of the example luer taken along section A-A illustrated in FIG. 3D.

### DETAILED DESCRIPTION

The following discussion omits or only briefly describes conventional features of luers that are apparent to those skilled in the art. Those of ordinary skill may thus recognize that other elements may be desirable and/or necessary to implement the devices, systems, and methods described herein. It is noted that various examples are described in detail with reference to the drawings. Reference to these various examples does not limit the scope of the claims attached hereto. Additionally, any examples set forth in this specification are intended to be non-limiting and merely set forth some of the many possible implementations for the appended claims. Further, particular features described herein can be used in combination with other described features in each of the various possible combinations and permutations. As such, it is understood that the detailed description is exemplary and explanatory only and is not restrictive of the broad inventive concepts upon which the examples disclosed herein are based.

Unless otherwise specifically defined herein, all terms are to be given their broadest reasonable interpretation. This includes meanings implied from the specification as well as meanings understood by those skilled in the art and/or as defined in dictionaries, treatises, etc.

It is noted that, as used in the specification and the appended claims, the singular forms “a,” “an” and “the” include plural referents unless otherwise specified. The terms “includes” and/or “including,” when used in this specification, specify the presence of stated features, elements, and/or components, but do not preclude the presence or addition of one or more other features, steps, operations, elements, components, and/or groups thereof.

Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then-described or as shown in the drawing figure under discussion. These relative terms are for convenience of description and normally are not intended to require a particular orientation in actuality. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The phrases “operatively” or “operably connected” indicates such an attachment, coupling or connection that allows the pertinent structures to operate as intended by virtue of that relationship.

Reference throughout the specification to “exemplary”, “one example”, “an example” or “some examples” means that a particular feature, structure, or characteristic described in connection with at least one example of the subject matter disclosed. Thus, the appearance of the phrases “in one example”, “in an example” or “in some examples” in various places throughout the specification is not necessarily referring to the same example. Further, the particular features, structures or characteristics of “one example”, “an example” or “some examples” may be combined in any suitable manner with each other to form additional examples of such combinations. It is intended that examples of the disclosed subject matter cover modifications and variations thereof. Terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Moreover, throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range. As used herein, the term “about” in reference to a measurable value, such as an amount, a temporal duration, and the like, is meant to encompass variations of plus or minus 20%, plus or minus 10%, plus or minus 5%, plus or minus 1%, and plus or minus 0.1% of the specified value, as such variations are appropriate.

The terms “proximal,” “distal,” “anterior,” “posterior,” “medial,” “lateral,” “superior,” and “inferior” are defined by their standard usage indicating a directional term of reference. For example, “proximal” refers to a position that is situated nearer to the center of a body or point of attachment or interest, while “distal” refers to a position that is situated away from the center of the body or point of attachment or interest. In another example, “anterior” refers to the front of a body or structure, while “posterior” refers to the rear of a body or structure, in relation to a relative viewpoint. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

Conventional luers coupled with tubing, such as tubing for aspirating particles and fluid, are prone to clogging. In particular, the connection point between a typical luer and tubing forms a rapid flow transition which tends to obstruct the flow path of the aspirated particles and fluid. Further, conventional luers are difficult to grip. As such, removing the luer from the tubing by hand to clear any blockages is typically an uncomfortable and/or onerous experience.

The examples of luer fittings described herein provide an improved flow path and smooth flow through the luer and engaged tubing, thereby reducing blockages within the luer. Moreover, the examples described herein provide luers having ergonomic surfaces that thereby improve user grip, comfort, and application of force when attaching or separating the luer from tubing or the handpiece. Examples of the disclosed luer fittings for phacoemulsification handpieces are described below with reference to the Figures.

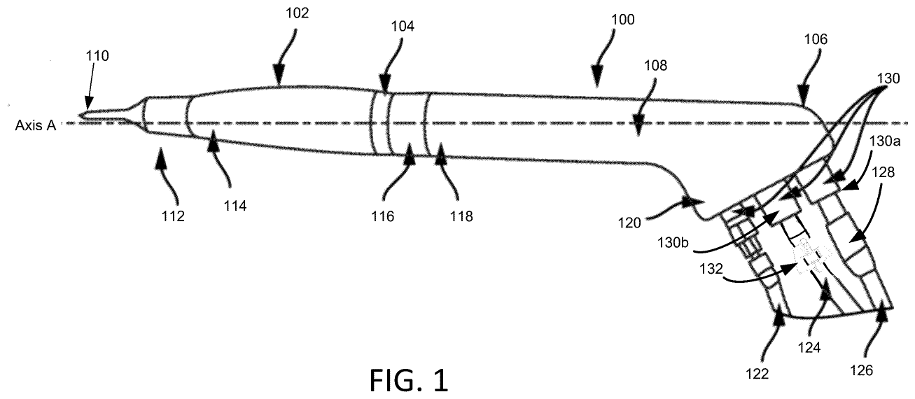

FIG. 1 illustrates an example handpiece 100 of a phacoemulsification system. In one or more cases, the handpiece 100 may have at least two segments, namely a proximal segment 108 and a distal segment 102. The proximal segment 108 and distal segment 102 may be referred to as the first segment 108 and the second segment 102, respectively.

The first segment 108 and second segment 102 may be coupled to each other. For example, the first segment 108 may be coupled with the second segment 102 via a first end 118 of the first segment 108 and a second end 104 of the second segment 102. The first segment 108 and second segment 102 may be coupled together by a coupler 116, such as, but not limited to a snap fit feature having male and female connectors. In one or more cases, the coupler 116 may have a swivel feature allowing the first segment 108 and the second segment 102 to rotate about an axis A independently of each other. In one or more cases, one or both of the first segment 108 and the second segment 102 are configured to rotate 360 degrees. In one or more other cases, the rotation may be limited to less than 360 degrees depending upon the freedom of movement and amount of control desired.

In one or more cases, the second segment 102 of handpiece 100 may have a needle 110 connected to a distal portion of the second segment 102. A sleeve 112 may be coupled with the handpiece 100 and at least partially surround the needle 110. The needle 110 and sleeve 112 may be separate components attachable to the second segment 102 or may be integrally coupled with the second segment 102. The first segment 108 includes tubing/cord management section 120 that includes one or more port/connectors 130. The location and position of the one or more port/connectors 130 helps manage the cords and/or tubing, such as cord 122 and tubing 124 and 126 connected thereto. For example, based on the location of the ports 130, the connected cords and/or tubing may lay or rest against a user's hand to keep the connected cords and/or tubing in a comfortable and convenient location.

The one or more port/connectors 130 may all be aligned or may exit the tubing/cord management section 120 at any angle to achieve an ergonomic handpiece. In one or more cases, the port/connector 130 are between 0 degrees and 90 degrees from the longitudinal Axis A, and preferably 0 degrees to 45 degrees. It is noted that handpiece 100 may include one or more of the same or similar features as, for example, handpieces 500, 600, 700, 800, and 900 as described in U.S. Pat. No. 11,266,384. As such, U.S. Pat. No. 11,266,384 is incorporated herein by reference in its entirety.

Tubing 124 and 126 may be connected to respective ports 130b and 130a via respective luer fitting, such as luer fittings 132 and 128. Luer fitting 128 and/or 132 may be any exemplary luer described herein. Tubing 124 and 126 may be formed of, by way of non-limiting example, polyvinyl chloride (PVC), plasticized PVC, or the like. Luer fitting 132 may be an irrigation luer-lock fitting, such as a female luer, configured to assure connection between tubing 124 and the handpiece 100.

In one or more cases, the tubing 124 may be an aspiration tube that couples together an aspiration channel of the handpiece 100, via the port 130b, and a collection receptacle. To couple the tubing 124 to the luer fitting 132, an adhesive, such as, but not limited to, cyclohexane adhesive, may be applied to a barb of the luer fitting 132, and the tubing 124 may be subsequently pushed over the barb of the luer fitting 132. A suction/vacuum may be applied to the tubing 124 to aspirate fluid and emulsified particles through handpiece 100 and into a collection receptacle (not shown) during a phacoemulsification procedure, such as is described in U.S. Patent Publication Number 2022/0133537. As such, U.S. Patent Publication Number 2022/0133537 is incorporated herein by reference in its entirety.

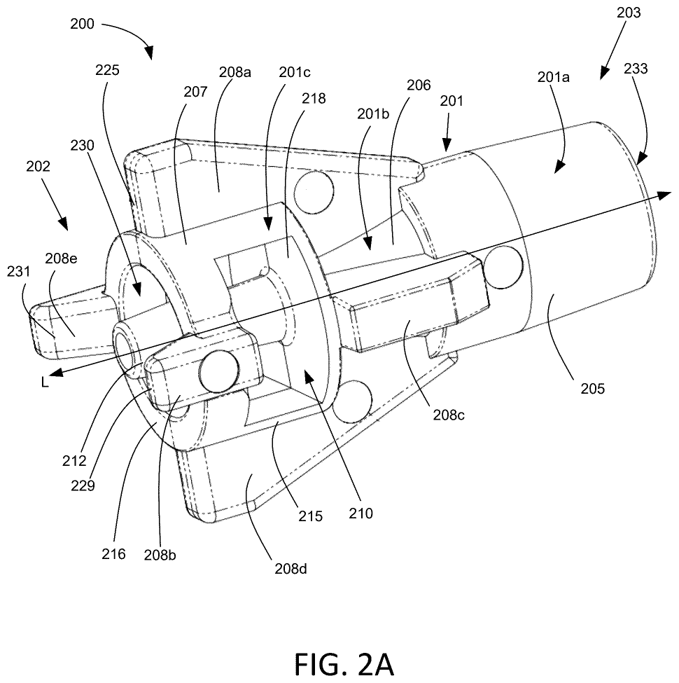

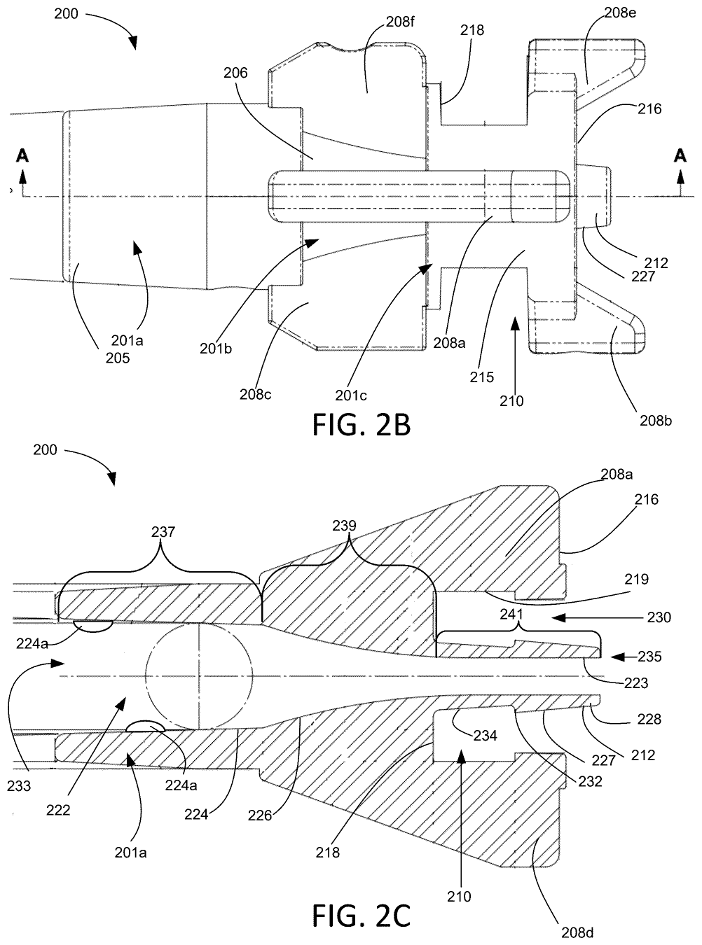

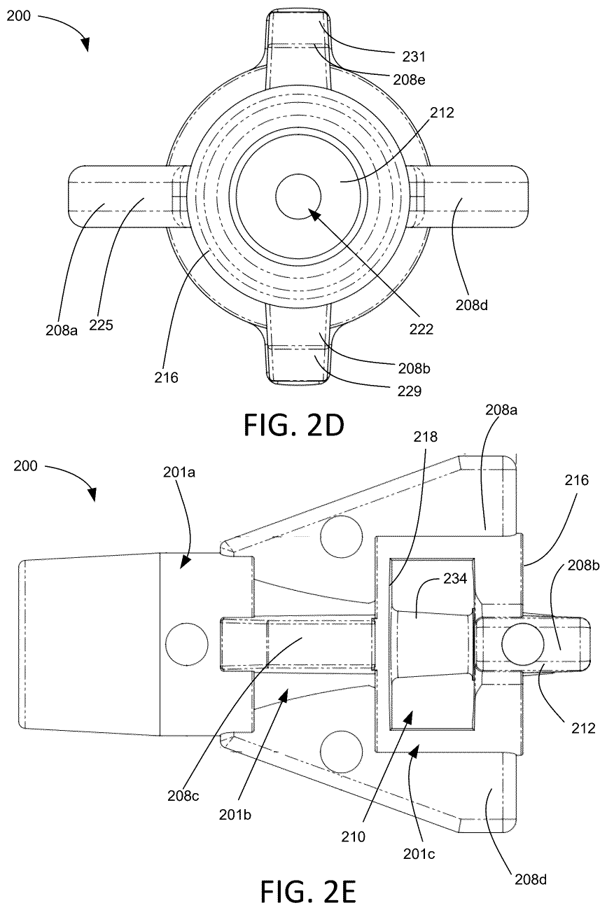

FIGS. 2A-2E illustrate various views of an example luer fitting 200. For example, FIG. 2A illustrates a perspective view of luer fitting 200. FIGS. 2B and 2E illustrate side views of the luer fitting 200. FIG. 2C illustrates a cross-sectional side view of the luer fitting 200 taken along section A-A illustrated in FIG. 2B. FIG. 2D illustrates a rear view of the luer fitting 200. Luer fittings 128 and/or 132 of FIG. 1 may include the same or similar features as those described for luer fitting 200.

The luer fitting 200 includes a rigid body 201 having a proximal end 202, a distal end 203 and a length L. The length L may be about 1 centimeter (cm) to 5 cm. The body 201 may include a distal or first region 201a, a central or second region 201b, and a proximal or third region 201c. The body regions 201a, 201b and 201c may be formed as a single unit, or they may be separable component parts. The luer fitting 200 may be, by way of non-limiting example, a female luer configured to securely connect tubing, such as tubing 124, to a port, such as port 130b, of the handpiece 100. In other examples, the luer fitting 200 may be a male luer fitting at one or more ends.

The first region 201a, second region 201b, and third region 201c include a tubular or hollow center that defines a passage 222. The passage 222 may be sized to pass a fluid (e.g. a liquid and/or a gas) and/or particles therethrough. For example, the luer fitting 200 may be connected to an aspiration port and tubing to aspirate fluid and emulsified particles from the handpiece 100 and into a collection receptacle during a phacoemulsification procedure. It is noted that although the luer fitting 200 is discussed in a configuration for aspiration during a phacoemulsification procedure, it should be appreciated that the luer fitting 200 is not limited to use in only phacoemulsification procedures but may also be used in any other desirable applications involving flow of a fluid and/or particles, such as coupling a port of an intravenous bag, medication bag, cryopreservation bag, and the like with tubing or other flow line.

The portion of the body 201 at first region 201a has an exterior surface 205 and may be any shape, such as a cylindrical member in which a portion of a distal end of the passage 222 is sized to couple to a port, such as port 130b, of the handpiece 100. In one or more cases, the first region 201a includes a female connection opening 233 configured to receive a portion of the port in the passage 222.

The portion of the body 201 at second region 201b couples the first region 201a to the third region 201c. The second region 201b has an exterior surface 206 and may be any shape, such as a conical shape, cylindrical shape, hexagonal shape, octagonal shape, or other like cylindrical or polygonal shape or shapes, or combinations thereof, and may add rigidity to the connection between the first region 201a and the third region 201c. In one or more cases, the exterior surface 206 of the second region 201b tapers from the first region 201a to the third region 201c. As such, the diameter of the exterior surface 206 of the second region 201b adjacent the first region 201a is larger than the diameter of the exterior surface 206 of the second region 201b adjacent the third region 201c.

The third region 201c has an exterior surface 207 and may also be any shape, for example such as a cylindrical shape, conical shape, octagonal shape, or other like cylindrical or polygonal shapes. The third region 201c has a proximal end face 216 and may include an opening to a recessed region 230 therein. An inner back wall 218 and an inner sidewall 219 form the general enclosure of the recessed region 230. The recessed region 230 may be sized and configured to house or surround at least a portion of a barb 212. The recessed region 230 may have an inner diameter sized and configured to receive an end of a tube, such as tubing 124 in FIG. 1. For example, the inner diameter of the recessed region 230 may be about 0.125 cm to 0.9 cm. The barb 212 may extend proximally from the inner back wall 218 within the recessed region 230. In one or more cases, the proximal end 228 of the barb 212 extends proximally beyond the proximal end face 216 of the third region 201c. In such cases, the extended portion of the proximal end 228 allows a user to more easily view the end of the barb 212 when positioning tubing (e.g. tubing 124) onto the barb 212. In one or more other cases, the proximal end 228 of the barb 212 is set back from the proximal end face 216, such that it remains within the recessed region 230. In yet one or more other cases, the proximal end 228 of the barb 212 is flush with proximal end face 216.

In one or more cases, the exterior surface 207 of the third region 201c includes one or more windows or openings 210, such that a portion of the recessed region 230 is exposed and viewable. The one or more openings 210 may allow a user to easily see inside the recessed region 230. For example, when positioning tubing onto the barb 212, the user may view through the one or more openings 210 when a distal end of the tubing 124 contacts the inner back wall 218. In some cases, the exterior surface has two opposing windows 210, thereby creating a transverse opening through the third body region 201c and permitting visualization of the barb 212 from both sides of luer fitting 200. In one or more other cases, the exterior surface 207 of the third region 201c is uniform and does not include any openings.

The passage 222 extends from the opening 233 at the distal end of the first region 201a to an opening 235 of the proximal end 228 of the barb 212. The opening 233 at the end of the first region 201a may have a greater diameter than the opening 235 of the proximal end 228 of the barb 212. The passage 222 forms an inner surface 224 that defines a pathway for fluid and/or particles to pass through from the handpiece 100 to the tubing 124. The passage 222 may be defined by a distal or first portion 237, a central or second portion 239, and a proximal or third portion 241. That is, the first portion 237 of the passage 222 may be located in the first region 201a of the body 201, the second portion 239 of the passage 222 may be located in the second region 201b of the body 201, and the third portion 241 of the passage 222 may be located in the barb 212 within the third region 201c of the body 201. For example, the first portion 237 of the passage 222 extends from the opening 233 at the distal end of the first region 201a to a distal end of the second region 201b. The second portion 239 of the passage 222 extends from the distal end of the second region 201b to the distal end of the third region 201c, which is also the location of the inner back wall 218. The third portion 241 of the passage 222 extends from the inner back wall 218 through the length of barb 212 to the opening 235 at the proximal end 228 of the barb 212.

In one or more cases, the first portion 237 of the passage 222 may have a uniform diameter along its length, or it may have a variable diameter along its length. In one or more other cases, the diameter of first portion 237 of the passage 222 may gradually reduce in size, such that the diameter of the passage 222 at the opening 233 may be greater than the diameter of the passage 222 at the proximal end of first portion 237. The second portion 239 of the passage 222 may serve to transition a larger diameter of the first portion 237 to a smaller diameter of the third portion 241 of the passage 222. For example, the inner surface 224 of the second portion 239 of the passage 222 may include a tapered, frustoconical, or curvilinear surface shape 226, such that the diameter of the second portion 239 of the passage 222 at its distal end is greater than the diameter of the second portion 239 of the passage 222 at its proximal end along its length. In some cases, the transition in diameter of the passage 222 from the first portion 237 to the second portion 239 is smooth and/or gradual. In some cases, the transition in diameter of the passage 222 from the first portion 237 to the second portion 239 is abrupt, such as a step down or forming a shoulder surface. In one or more cases, the third portion 241 of the passage 222 may have a uniform diameter along its length, or it may have a variable diameter along its length. In one or more other cases, the diameter of third portion 241 of the passage 222 may gradually reduce in size, such that the diameter of the third portion 241 of the passage 222 from the inner back wall 218 is greater than the diameter of the opening 235 of the proximal end 228 of the barb 212.

In one or more cases, the inner surface 224 of the passage 222 may be smooth at all or any portion of first, second and third portions 237, 239 and 241. In one or more other cases, the inner surface 224 of the passage 222 may be contoured, or otherwise include one or more protruding structures (protrusions) 224a, at all or any portion of first, second and third portions 237, 239 and 241 to improve and/or modify the flow of liquid and/or particles through the passage 222. In some cases, protruding structures 224a are configured to reduce the frequency of particles or material becoming stuck or lodged when traveling through passage 222. Exemplary protruding structures 224a may include a rib, ridge, bump, trough, groove, step, shoulder, and the like. For example, the inner surface 224 of the passage 222 may be rifled. In one or more other cases, portions of the inner surface 224 of the passage 222 may be smooth, and other portions of the inner surface 224 of the passage 222 may be contoured. For example, the inner surface 224 of the first portion 237 and the second portion 239 of the passage 222 may be contoured, and the inner surface 224 of the third portion 241 is smooth.

The barb 212 may be a generally cylindrical body having an inner surface 223 forming the third portion 241 of passage 222, and having an outer surface 227. The inner surface 223 of the barb 212 defines an inner diameter of the barb 212. For example, the inner diameter of the barb 212 may be about 0.06 cm to 0.6 cm. The outer surface 227 of the barb 212 defines an outer diameter of the barb 212. The area of the barb 212 between the inner and outer diameter of the barb 212 defines the thickness of the barb 212. For example, the thickness of the barb 212 may be about 0.05 cm to 0.8 cm. The length of the barb 212 may be, for example, about 0.125 cm to 0.65 cm. The barb 212 includes a neck 234 at its distal end that extends proximally from inner back wall 218 towards one or more shoulders 232 on the outer surface 227 of the barb 212.

The one or more shoulders 232 may be disposed on the outer surface 227 of the barb 212 in a variety of sizes, shapes, and patterns to facilitate friction and a secure fit of the tubing 124 engaging the barb 212 of luer fitting 200. In one or more cases, the shoulder 232 may be one or more annular ridges that fully extend around the circumference of the outer surface 227. The outer diameter of the barb 212 at the shoulder 232 may be larger than the outer diameter along the neck 234 of the barb 212. In some cases, the outer diameter of the barb 212 at the shoulder 232 may be larger than the outer diameter at the proximal end 228 of the barb, such that the outer surface 227 tapers from the shoulder 232 to the opening 235 of the proximal end 228 of the barb 212. In one or more other cases, the outer surface 227 of the proximal end 228 of the barb 212 is cylindrically shaped, such that the diameter of the outer surface 227 is uniform from the shoulder 232 to the opening 235 of the proximal end 228 of the barb 212. In one or more other cases, the one or more shoulders 232 may be a series of tabs that protrude outwardly from the outer surface 227 of the barb 212 and may be circumferentially disposed and spaced apart from one another around the outer surface 227 of the barb 212 along its length. In one or more other cases, the barb 212 includes a plurality of shoulders 232 arranged as concentric rings along the length L of the barb 212. In yet one or more other cases, the barb 212 includes a plurality of shoulders 232 arranged in sequence or randomly along the length of the barb 212, in which at least two shoulders are each formed as a series of tabs that protrude from the outer surface 227 of the barb 212 and are circumferentially disposed and spaced apart from one another around the outer surface 227 of the barb 212. In some cases, the series of shoulder tabs may be linearly arranged while in other cases, they may be offset.

The outer diameter of the barb 212 is sized such that tubing is snuggly secured onto the barb 212 when coupled with the barb 212. For example, the outer diameter of the barb 212 may be sized to be slightly larger than the inner diameter of the tubing, such that the tubing expands as the tubing is pressed onto and over the barb 212, thus creating a tighter and more snug fit. That is, the compliant tubing expands over the shoulder 232 and conforms to the outer diameter of the shoulder 232 and/or neck 234 of the barb 212. To further couple the tubing 124 to the luer fitting 200, an adhesive, such as, but not limited to, cyclohexane adhesive, may be applied to the barb 212, and the tubing 124 may be subsequently pressed onto the barb 212. The tubing may be pressed onto the barb 212 until a distal end of the tubing contacts the wall 218 or is positioned beyond the shoulder 232 and around a portion of the neck 234. The shoulder 232 may contact the inner surface of the tubing 124 to increase friction between the barb 212 and tubing 124, thereby creating a tighter fit.

The luer fitting 200 may include one or more protrusions, such as protrusions 208a-208f, that extend outward from one or more exterior surfaces 205, 206 and 207 of the body 201. The one or more protrusions may have the same rigidity as the body 201, and in some cases, they may have more rigidity or less rigidity than body 201. The one or more protrusions may be disposed around any portion of the body 201 of luer fitting 200 along the length L. In one or more cases, a protrusion, such as protrusion 208a, may extend outwards from the exterior surface that includes multiple regions of the body 201 of luer fitting 200, such as the first region 201a, the second region 201b, and the third region 201c. In one or more cases, a protrusion, such as protrusion 208b, may extend outwards from only one region of the body 201 of luer fitting 200, such as from the exterior surface 207 of the third region 201c. In one or more cases, a protrusion, such as protrusion 208c, may extend outwards from only two regions of the body 201 of luer fitting 200, such as from the exterior surface 205 of the first region 201a and from the exterior surface 206 of the second region 201b. As such, a protrusion may extend from the exterior surface of several regions of the body 201 of luer fitting 200, thereby connecting the several body regions and acting as a structural support of the luer fitting 200. Moreover, by adding structural support via one or more of protrusions across one or more of the first region 201a, the second region 201b, and the third region 201c, the one or more protrusions and exterior surface 207 of the third region 201c form a shroud configured to protect the barb 212. For example, the shroud may protect the barb 212 from breaking, being bent, or otherwise damaged from inadvertent sideloading when positioning tubing onto the barb 212. In another example, the shroud may protect the barb 212 from breaking, being bent, or otherwise damaged due to the weight of the tubing or the tubing being pulled away from the barb 212.

In some cases, the one or more protrusions may serve as gripping features, such that a user may have one or more points of contact on the exterior surface of the luer fitting 200 that allow the user to easily grasp the luer fitting 200 when removing the luer fitting 200 from, for example, the handpiece 100 or tubing 124. In some cases, the one or more protrusions may provide added structural support and/or leverage to the exterior surface of the luer fitting 200, such that the user may press against one or more of the protrusions to twist and/or push/pull the luer fitting 200 on/off a port of the handpiece 100 without deforming or otherwise damaging the shape of the luer fitting 200 or the ports 130.

Any protrusion may extend outwardly from the outer surface of the body 201 by about 0.05 cm to 0.07 cm. In some cases, two or more protrusions extend the same distance from the exterior surface of the luer fitting 200. In some cases, two or more protrusions each extend a different distance from the exterior surface of the luer fitting 200. For example, protrusions 208a and 208d may extend radially outwards from the body 201 farther than that of protrusions 208b and 208e, as illustrated in FIG. 2D.

In one or more cases, multiple protrusions, such as protrusions 208b and 208c, may extend along the length L of the body 201 in a linear fashion, such that protrusion 208b and protrusion 208c are aligned with one another longitudinally. In one or more other cases, the multiple protrusions may be offset from one another about the circumference of the body 201. For example, when viewed down the length L of the body 201, a first protrusion may be positioned at 0° about the circumference of the body 201, and a second protrusion may be positioned at 45° about the circumference of the body 201. In one or more cases, the multiple protrusions, such as protrusions 208b and 208c, may be spaced apart from one another along the length L of the body 201 to accommodate the opening 210.

The one or more protrusions may be shaped to prevent or mitigate sideloading that may deform or damage the body 201 of the luer fitting 200. Additionally or alternatively, the shape of one or more protrusions may aid, such as by providing a more suitable angle for force application and/or an increased surface area for force application, a user when grasping, pushing, pulling, and/or turning the luer fitting 200. For example, when the luer fitting 200 is coupled to a port of a handpiece, such as port 130b of handpiece 100, a user may grasp onto protrusions 208a and 208d while turning and/or pulling the luer fitting 200 away from port 130b. The one or more protrusions may be formed in in any shape, such as, by way of non-limiting example, a wing shape. In one or more cases, the wing-shaped protrusion, such as protrusion 208a, may taper downwards towards the first region 201a (i.e. the distance the protrusion extends from the exterior surface of the body 201 changes along the length of the protrusion). In some cases, the proximal end of the protrusion, such as the proximal end 225 of protrusion 208a, may be set back from the proximal end face 216 of the third body region 201c. In other cases, the proximal end 225 of protrusion 208a may reside on the same planar surface of the proximal end face 216. In one or more cases, a protrusion may include a proximal end portion that extends beyond one or both of the proximal end face 216 of the third body region 201c and/or the proximal end 228 of the barb 212. For example, protrusion 208b and/or 208e may include a proximal end portion 229 and/or 231, respectively, that extends proximally beyond the proximal end face 216. The portions of the protrusions that extend proximally beyond one or both of the proximal end face 216 and the proximal end of the barb 212 may shroud the proximal end 228 of the barb 212, thereby preventing or mitigating the proximal end 228 of the barb 212 from inadvertently being damaged.

In one or more cases, the luer fitting 200 may include two or more protrusions circumferentially disposed around the luer fitting 200. In some cases, the two or more protrusions may be equally spaced apart from one another about the circumference of the body 201. In one or more cases, the protrusions may be sized and spaced apart from one another so as to have a lower profile and not interfere with an adjacent luer, port or other system component when coupled with a handpiece.

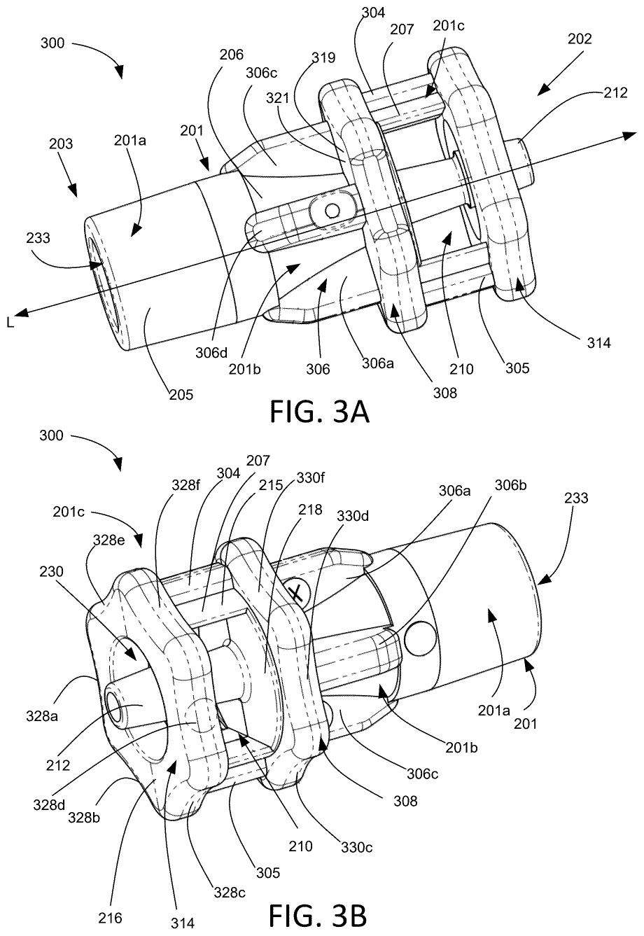

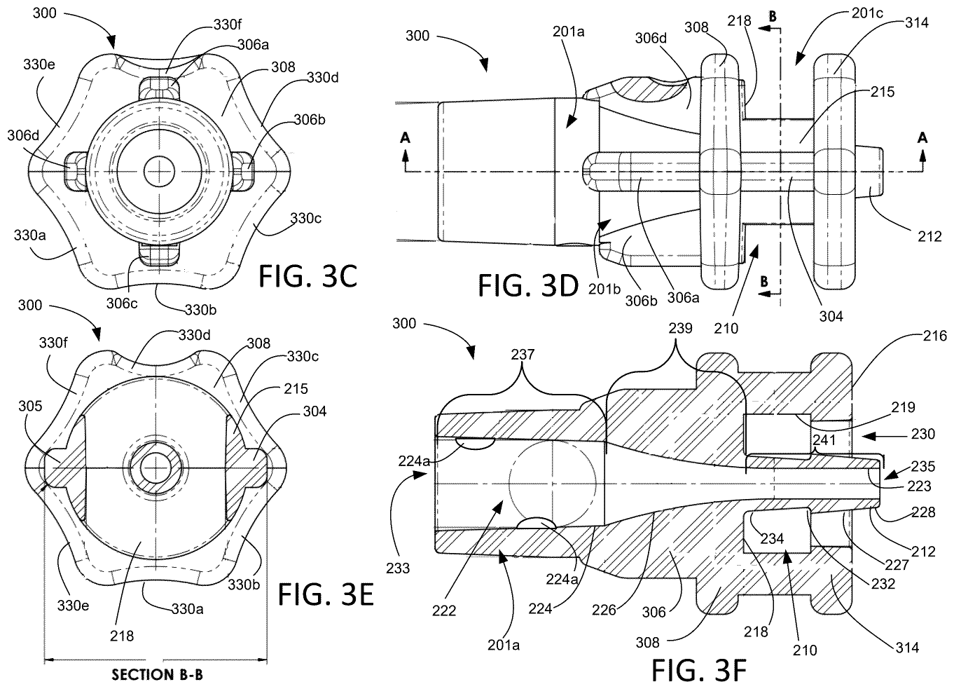

FIGS. 3A-3F illustrate various views of another exemplary luer fitting 300. The luer fitting 300 illustrated in FIGS. 3A-3F is distinguishable from that of the luer fitting 200 illustrated in FIGS. 2A-2E at least in that the example luer fitting 300 includes one or more gripping flanges, such as flanges 308 and 314, about the exterior surface of the luer fitting 300. Further, the like reference numerals of FIGS. 2A-3F represent like parts and assemblies, and as such, a description of these parts and assemblies is not repeated.

FIGS. 3A and 3B illustrate perspective views of a luer fitting 300, having a different profile of protrusions as compared to the examples of FIGS. 2A-2E. Further, FIG. 3C illustrates a rear view of the luer fitting 300. FIG. 3D illustrates a side view of the luer fitting 300. FIG. 3E illustrates a cross-sectional side view of the luer fitting 300 taken along section B-B illustrated in FIG. 3D. FIG. 3F illustrates a cross-sectional side view of the luer fitting 300 taken along section A-A illustrated in FIG. 3D. Luers 128 and/or 132 of FIG. 1 may include the same or similar features as those described for luer fitting 300. Like exemplary luer fitting 200, luer fitting 300 is not limited to use in only phacoemulsification procedures but may be used in other applications, such as coupling a port of an intravenous bag, medication bag, cryopreservation bag, and the like with tubing for the respective application.

With regard to the protrusions, luer fitting 300 may include one or more protrusions in the form of flanges, such as flanges 308 and 314. A flange may be shaped and positioned along the luer fitting 300 to prevent or mitigate sideloading that may deform or damage the body 201 of the luer fitting 300. Further, the luer fitting 300 may include one or more longitudinal support protrusions, such as support protrusions 304, 305 and 306. For example, the support protrusions 306 may be used in combination with the one or more flanges 308 and/or 314 to reduce the overall profile of the luer fitting 300 while preventing or mitigating sideloading that may deform or damage the body 201 of the luer fitting 300. The one or more flanges 308 and/or 314 may serve as gripping features, such that a user may have one or more points of contact on the luer fitting 300 that allow the user to easily grasp the luer fitting 300 when removing the luer fitting 300 from or attaching the luer fitting 300 to, for example, the handpiece 100 or tubing 124. For example, the flanges 308 and 314 and support protrusions 306 provide added structural support to the luer fitting 300, such that the user may press against one or both of the flanges 308 and 314 to twist, push, and/or pull the luer fitting 300 off a port of the handpiece 100 without deforming or otherwise damaging the shape of the luer fitting 300 or the ports 130.

The support protrusions 306 extend outward from an exterior surface of the body 201 at one or more of the first region 201a, the second region 201b, and the third region 201c. For example, support protrusions 306a, 306b, 306c, and 306d extend outward from an exterior surface of the body 201 at the first region 201a and the second region 201b along its length. The one or more support protrusions 306 may be disposed around the luer fitting 300. The one or more flanges extend outward from the body 201 at one or more of the first region 201a, the second region 201b, and the third region 201c. For example, a flange, such as flange 314, extends outward from a portion of the body 201 that includes the third region 201c. In another example, a flange, such as flange 308, extends outward from portions of one or both of the second region 201b and the third region 201c.

A support protrusion, such as support protrusions 306a, 306b, 306c, and 306d, is a rigid body that extends from an exterior surface of the body 201 along the length L. In one or more cases, a protrusion, such as protrusion 306a, may protrude from the exterior surface of several regions of the luer fitting 300, thereby connecting the several regions and acting as a structural support of the luer fitting 300. For example, the protrusion 306a may protrude from the body 201 of the luer fitting 300 that includes at least a portion of second region 201b and at least a portion of the surface of the first region 201a. Further, an end of the support protrusion may interface with a rear surface of a flange. For example, an end 321 of the support protrusion 306a may interface with a rear surface 319 of the flange 308. As such, the arrangement of protrusions 306 about the body 201 of the luer fitting 300 may increase the structural integrity of the body 201, and in particular the portion of the body 201 that includes the second region 201b. The support protrusion may be formed in a variety of shapes, such as, by way of non-limiting example, a wing shape, or a rectangular shape with a chamfered corner.

A flange protrusion, such as flange 308 and flange 314, may be a rigid body having, for example, but not limited to, a rounded or unrounded hexagonal shape. The flanges 308 and 314 may each include a plurality of gripping features. A gripping feature may be formed in a contoured and smooth surface. A series of gripping features on a flange may be of a consistent size of raised and lowered or concaved features, or some or all of the gripping features may have different heights, concavities, and shapes, thereby providing an ergonomic grip. The series of gripping features may be disposed around an outer perimeter of the outer surface of each flange. For example, flange 308 may include gripping features 330a, 330b, 330c, 330d, 330e, and 330f. In another example, flange 314 may include gripping features 328a, 328b, 328c, 328d, 328e, and 328f. In one or more cases, each of the gripping features may extend perpendicularly across the flange, such that the series of gripping features may form straight knurls around the outer perimeter of the flange. In one or more other cases, each of the gripping features may extend diagonally across the flange, such that the series of gripping features may form straight knurls around the outer perimeter of the flange. The gripping features of each flange 308, 314 provide several points of contact on the luer fitting 300 that allow the user to easily grasp the luer fitting 300 when removing the luer fitting 300 from, for example, the handpiece 100 or tubing 124. For instance, the gripping features may be positioned around each flange 308, 314 to facilitate a pincer grip. The gripping features may maximize the grip area of the luer fitting 300, in particular, when a user is wearing gloves.

It is noted that although each of flanges 308, 314 are hexagonal and include six gripping features as shown, it should be understood that each flange may be any shape and include more than six gripping features or less than six gripping features. Further, in some cases, the flanges 308 and 314 may include the same number of gripping features. In other cases, a first flange may include a higher number of gripping features than a second flange. In one or more cases, the gripping features of one flange may be aligned with the gripping features of the other flange. For example, gripping feature 328a may be aligned with gripping feature 330a, gripping feature 328b may be aligned with gripping feature 330b, gripping feature 328c may be aligned with gripping feature 330c, gripping feature 328d may be aligned with gripping feature 330d, gripping feature 328e may be aligned with gripping feature 330e, and gripping feature 328f may be aligned with gripping feature 330f. In one or more other cases, the gripping features of one flange may be offset from the gripping features of the other flange.

Flanges 308 and 314 may be disposed on opposite sides, or opposite ends, of the third body region 201c. The third body region 201c may include one or more protrusions, such as structures 304 and 305, disposed between flanges 308 and 314. The structures 304 and 305 may each extend from the exterior surface 207. Further, the structures 304 and 305 may each extend across the exterior surface and contact the interior facing surfaces of the flanges 308 and 314. The structures 304 and 305 may provide structural integrity to the luer fitting 300, such that the luer fitting 300 maintains its shape when subjected to an external force. The exterior surface 207 of the third body region 201c and the opposing inside surfaces of the flanges 308 and 314 define the recessed region 230 sized to house at least a portion of the barb 212. Thus, exterior surface 207 of the third body region 201c and flanges 308 and 314 may form a shroud configured to protect the barb 212.

In one or more cases, the luer fitting 200 or 300 is formed having a unibody or single unit construction. In one or more other cases, the luer fitting 200 or 300 is an assembly of separate component parts. The luer fitting 200 or 300 may be formed using one or a variety of manufacturing processes, such as, but not limited to, injection molding, 3D printing, and the like. For example, the luer fitting 200 may be formed in two or more pieces that are fastened to one another by being screwed, snapped, or bonded (e.g., using welding or adhesive) to one another. The luer fitting 200 or 300 may be formed of material, such as, but not limited to, thermoplastics (e.g., acrylonitrile butadiene styrene (ABS)), nylon, polypropylene, polyethylene, polycarbonate, polyvinylidene fluoride (PVDF), metal suitable for medical applications (e.g., surgical steel), and other like materials that provide secure and leak-proof connections. In one or more cases, the barb 212 and the body 215 may have the same rigidity. In one or more other cases, the barb 212 may be more compliant than that of the body 215. That is, the barb 212 may have a degree of flexibility such that the barb 212 may bend but not break when the luer fitting 200 is coupled with or removed from tubing. In one or more other cases, the third body region 201c may be more compliant than that of the barb 212. For instance, the barb 212 and the third body region 201c may be formed having different thicknesses and/or different materials, thereby altering the compliancy of the barb 212 and the compliancy of the third body region 201c.

### EXAMPLES

### Example 1

A luer fitting, comprising: a body (201) having a proximal region (201c), a distal region (201a), and a length (L) therebetween, the body further comprising: a recessed region (230) within a portion of the length of the body at the proximal region, the recessed region having a proximal end opening, an inner back wall (218) and an inner circumferential sidewall (219) between the inner back wall and the proximal end opening; a barb (212) extending proximally from the inner back wall of the recessed region; and a passage (222) between a first opening (233) at the distal region of the body to a second opening (235) at a proximal end of the barb, the passage forming an inner surface (224) and having a first inner diameter at or near the first opening at the distal region opening that is greater than a second inner diameter at or near the inner back wall of the recessed region, wherein the transition between the first inner diameter and the second inner diameter is curvilinear.

### Example 2

A luer fitting, comprising: a body (201) having a proximal region (201c), a central region (201b), a distal region (201a), and a length (L) therebetween forming an exterior surface, the body further comprising: a recessed region (230) within a portion of the length of the body at the proximal region, the recessed region having a proximal end opening, an inner back wall (218) and an inner circumferential sidewall (219) between the inner back wall and the proximal end opening; a plurality of protrusions (208a, 208b, 208c, 208d, 208e, 208f) circumferentially disposed around and extending from the exterior surface of the body at the proximal region; a barb (212) extending proximally from the inner back wall of the recessed region; and a passage (222) between a first opening (233) at the distal region of the body to a second opening (235) at a proximal end of the barb, the passage forming an inner surface (224) and having a first inner diameter at or near the first opening at the distal region opening that is greater than a second inner diameter at or near the inner back wall of the recessed region, wherein the transition between the first inner diameter and the second inner diameter is curvilinear.

### Example 3

A luer fitting, comprising: a body (201) having a proximal region (201c), a central region (201b), a distal region (201a), and a length (L) therebetween forming an exterior surface, the body further comprising: a recessed region (230) within a portion of the length of the body at the proximal region, the recessed region having a proximal end opening, an inner back wall (218) and an inner circumferential sidewall (219) between the inner back wall and the proximal end opening; a first flange (314) extending from the exterior surface at the proximal end and a second flange (308) extending from the exterior surface at the proximal region and the central region; a barb (212) extending proximally from the inner back wall of the recessed region; and a passage (222) between a first opening (233) at the distal region of the body to a second opening (235) at a proximal end of the barb, the passage forming an inner surface (224) and having a first inner diameter at or near the first opening at the distal region opening that is greater than a second inner diameter at or near the inner back wall of the recessed region, wherein the transition between the first inner diameter and the second inner diameter is curvilinear.

### Example 4

The luer fitting according to any of examples 1-3, wherein the inner circumferential sidewall (219) of the recessed region (230) comprises at least one opening (210).

### Example 5

The luer fitting according to either of examples 1, 3, and 4, further comprising a plurality of protrusions (208a, 208b, 208c, 208d, 208e, 208f, 306a, 306b, 306c, and 306d) circumferentially disposed around and extending from an exterior surface of the body (201).

### Example 6

The luer fitting according to any of examples 1-5, wherein a protrusion (208a, 208b, 208c, 208d, 208e, 208f, 306a, 306b, 306c, and 306d) of the plurality of protrusions comprises a wing shape.

### Example 7

The luer fitting according to any of examples 1-6, wherein one or more protrusions (208b and 208e) of the plurality of protrusions extends beyond at least one of the proximal end of the barb or the proximal end opening of the recessed region.

### Example 8

The luer fitting according to any of examples 1, 2, and 4-7, wherein a first protrusion (208b, 208e) extends from the exterior surface at the proximal region of the body, and wherein a second protrusion (208b, 208f) is linearly aligned with the first protrusion and extends from the exterior surface at a central region (201b) of the body and a portion of the distal region, the central region being disposed between the proximal region and the distal region.

### Example 9

The luer fitting according to any of examples 1, 2, and 4-7, wherein a first protrusion (208b, 208e) extends from the exterior surface at the proximal region of the body, and wherein a second protrusion (208b, 208f) is linearly aligned with the first protrusion and extends from the exterior surface at a central region (201b) of the body, the central region being disposed between the proximal region and the distal region.

### Example 10

The luer fitting according to any of examples 1 and 4-8, further comprising a first flange (314) extending from the exterior surface at the proximal region and a second flange extending from the exterior surface at a central region of the body.

### Example 11

The luer fitting according to any of examples 1, 4-6, and 10, further comprising a support protrusion (304) disposed between the first flange and the second flange and extends along the length of the central region of the body.

### Example 12

The luer fitting according to any of examples 1, 4-6, 10, and 11, further a support protrusion (306a) extending from the exterior surface at the central region and a portion of the distal region, wherein a portion of the support protrusion interfaces with a rear surface of the second flange.

### Example 13

The luer fitting according to any of examples 1, 3-6, 10, and 12, wherein the first flange comprises a series of contoured surfaces (328a, 328b, 328c, 328d, 328e, 328f) disposed around an outer perimeter of the first flange.

### Example 14

The luer fitting according to any of example 1-13, wherein the proximal end of the barb extends to or beyond the proximal end opening of the recessed region.

### Example 15

The luer fitting according to any of example 1-14, wherein the barb (212) comprises a surface (227) that tapers from a shoulder (232) circumferentially disposed around the barb to the proximal end (228) of the barb.

### Example 16

The luer fitting according to any of example 1-15, wherein the inner surface (224) of the passage includes one or more protrusions (224a).

### Example 17

The luer fitting according to any of example 1-16, wherein the sidewall (215) of the recessed region (230) is more rigid than the barb (212).

### Example 18

The luer fitting according to any of example 1-17, wherein the body further comprises a central region (201b) disposed between the proximal region and the distal region, wherein the central region comprises a frustoconical shape in which a first end of the central region is adjacent the distal region and the second end of the central region is adjacent the proximal region, and wherein the first end of the central region comprises a larger diameter than the second end of the central region.

### Example 19

The luer fitting according to any of example 1-18, wherein the proximal region, the distal region, the length of the body, and the barb comprise a unibody construction.

Although the examples described herein mainly address phacoemulsification procedures, the methods and systems described herein can also be used in other medical applications. Further, the various examples described above are provided by way of illustration only and should not be construed to limit the claims attached hereto. Those skilled in the art will readily recognize various modifications and changes that may be made without following the examples and applications illustrated and described herein, and without departing from the spirit and scope of the following claims.

What is claimed is:

1. A luer fitting, comprising:
a body having a proximal region, a distal region, and a length therebetween, the body further comprising:
a recessed region within a portion of the length of the body at the proximal region, the recessed region having a proximal end opening, an inner back wall and an inner circumferential sidewall between the inner back wall and the proximal end opening;
a barb extending proximally from the inner back wall of the recessed region; and
a passage between a first opening at the distal region of the body to a second opening at a proximal end of the barb, the passage forming an inner surface and having a first inner diameter at or near the first opening at the distal region opening that is greater than a second inner diameter at or near the inner back wall of the recessed region, wherein the transition between the first inner diameter and the second inner diameter is curvilinear.

2. The luer fitting of claim 1, wherein the proximal region, the distal region, the length of the body, and the barb comprise a unibody construction.

3. The luer fitting of claim 1, wherein the inner circumferential sidewall of the recessed region comprises at least one opening.

4. The luer fitting of claim 1, further comprising a plurality of protrusions circumferentially disposed around and extending from an exterior surface of the body.

5. The luer fitting of claim 4, wherein a protrusion of the plurality of protrusions comprises a wing shape.

6. The luer fitting of claim 4, wherein one or more protrusions of the plurality of protrusions extends beyond at least one of the proximal end of the barb or the proximal end opening of the recessed region.

7. The luer fitting of claim 4, wherein a first protrusion extends from the exterior surface at the proximal region of the body, and wherein a second protrusion is linearly aligned with the first protrusion and extends from the exterior surface at a central region of the body and a portion of the distal region, the central region being disposed between the proximal region and the distal region.

8. The luer fitting of claim 4, wherein a first protrusion extends from the exterior surface at the proximal region of the body, and wherein a second protrusion is linearly aligned with the first protrusion and extends from the exterior surface at a central region of the body, the central region being disposed between the proximal region and the distal region.

9. The luer fitting of claim 1, further comprising a first flange extending from an exterior surface of the proximal region and a second flange extending from the exterior surface of the body at a central region of the body.

10. The luer fitting of claim 9, further comprising a support protrusion disposed between the first flange and the second flange and extends along the length of the central region of the body.

11. The luer fitting of claim 9, further comprising a support protrusion extending from the exterior surface at the central region and a portion of the distal region, wherein a portion of the support protrusion interfaces with a rear surface of the second flange.

12. The luer fitting of claim 9, wherein the first flange comprises a series of contoured surfaces disposed around an outer perimeter of the first flange.

13. The luer fitting of claim 1, wherein the proximal end of the barb extends to or beyond the proximal end opening of the recessed region.

14. The luer fitting of claim 1, wherein the barb comprises a surface that tapers from a shoulder circumferentially disposed around the barb to the proximal end of the barb.

15. The luer fitting of claim 1, wherein the inner surface of the passage includes one or more protrusions.

16. The luer fitting of claim 1, wherein the sidewall of the recessed region is more rigid than the barb.

17. The luer fitting of claim 1, wherein the body further comprises a central region disposed between the proximal region and the distal region, wherein the central region comprises a frustoconical shape in which a first end of the central region is adjacent the distal region and the second end of the central region is adjacent the proximal region, and wherein the first end of the central region comprises a larger diameter than the second end of the central region.

18. A luer fitting, comprising:
a body having a proximal region, a central region, a distal region, and a length therebetween forming an exterior surface, the body further comprising:
a recessed region within a portion of the length of the body at the proximal region, the recessed region having a proximal end opening, an inner back wall and an inner circumferential sidewall between the inner back wall and the proximal end opening;
a plurality of protrusions circumferentially disposed around and extending from the exterior surface of the body at the proximal region;
a barb extending proximally from the inner back wall of the recessed region; and
a passage between a first opening at the distal region of the body to a second opening at a proximal end of the barb, the passage forming an inner surface and having a first inner diameter at or near the first opening at the distal region that is greater than a second inner diameter at or near the inner back wall of the recessed region, wherein the transition between the first inner diameter and the second inner diameter is curvilinear.

19. A luer fitting, comprising:
a body having a proximal region, a central region, a distal region, and a length therebetween forming an exterior surface, the body further comprising:
a recessed region within a portion of the length of the body at the proximal region, the recessed region having a proximal end opening, an inner back wall and an inner circumferential sidewall between the inner back wall and the proximal end opening;
a first flange extending from the exterior surface at the proximal end and a second flange extending from the exterior surface at the proximal region and the central region;
a barb extending proximally from the inner back wall of the recessed region; and
a passage between a first opening at the distal end of the body to a second opening at a proximal end of the barb, the passage forming an inner surface and having a first inner diameter at or near the first opening at the distal end opening that is greater than a second inner diameter at or near the inner back wall of the recessed region, wherein the transition between the first inner diameter and the second inner diameter is curvilinear.
